(19) United States
(12) Patent Application Publication
(10) Pub. No.: US 2018/0344625 A1
(43) Pub. Date: Dec. 6, 2018

(54) Oral Care Products and Methods

(71) Applicant: MGE Holdings LLC, Beverly Hills, CA (US)

(72) Inventors: Kourosh Maddahi, Beverly Hills, CA (US); Hessam Nowzari, Beverly Hills, CA (US)

(73) Assignee: MGE Holdings LLC, Beverly Hills, CA (US)

(21) Appl. No.: 16/057,607

(22) Filed: Aug. 7, 2018

Related U.S. Application Data
(63) Continuation-in-part of application No. 15/850,655, filed on Dec. 21, 2017.
(60) Provisional application No. 62/552,650, filed on Aug. 31, 2017.
(60) Provisional application No. 62/465,536, filed on Mar. 1, 2017.
(60) Provisional application No. 62/437,100, filed on Dec. 21, 2016.

(51) Int. Cl. A61K 8/9789; A61Q 11/00; A61K 8/20; A61K 8/9794; A61K 8/34; A61K 8/25

(52) U.S. Cl. CPC A61K 8/9789; A61Q 11/00; A61K 8/25; A61K 8/9794; A61K 8/345; A61K 8/20

(57) ABSTRACT

Methods of increasing saliva volume in the oral cavity of persons having xerostomia by administering an oral care product (a dentifrice or a mouthwash) that consists essentially of (i) a sea salt, preferably Dead Sea salt, (ii) cayenne pepper oil; (iii) grape seed oil, (iv) coconut oil, and (v) preferably one or both of xylitol and/or aloe vera leaf juice.

### CROSS-REFERENCE TO RELATED APPLICATIONS

This application is a continuation-in-part of pending U.S. patent application Ser. No. 15/850,655, filed Dec. 21, 2017. U.S. patent application Ser. No. 15/850,655 claims the benefit of the following three provisional patent applications: (i) U.S. Provisional Application No. 62/437,100, filed Dec. 21, 2016; (ii) U.S. Provisional Application No. 62/465,536, filed Mar. 1, 2017; (iii) U.S. Provisional Application No. 62/552,650 filed Aug. 31, 2017.

### STATEMENT REGARDING FEDERALLY SPONSORED RESEARCH OR DEVELOPMENT

Not applicable.

### FIELD OF INVENTION

The present invention relates to compositions and methods for improving the appearance and health of teeth and gums with oral care products containing naturally-derived ingredients.

### BACKGROUND OF INVENTION

The therapeutic and medicinal benefits of Dead Sea salts have been reported in the scientific literature, typically in connection with diseases of the skin and joints. See, e.g., Uriel Katz et al., “Scientific Evidence of the Therapeutic Effects of Dead Sea Treatments: A Systematic Review,” Seminars in Arthritis and Rheumatism, Vol. 42, No. 2 (October 2012), pp. 186-200, citing Z. Even-Paz, J. Shani, “The dead sea and psoriasis: Historical and geographic background,” Int J Dermatol, Vo. 28, No. 1 (1989), pp. 1-9 (345 g of mineral per liter (34.5% or 34.5 g/100 mL); Id. citing S. Sukenik et al., “The Dead Sea—a unique resort for patients suffering from joint diseases,” Harefuah, Vol. 149, No. 3 (2010), pp. (175-179)(180 to 215 g of mineral per liter). Dan Buskila et al., “Balneotherapy for Fibromyalgia at the Dead Sea,” Rheumatol Int, Vol. 20 (2001), pp. 105-108.

The water of the Dead Sea contains concentrated salts other than NaCl—including, but not limited to, MgCl₂, CaCl₂), KCl, and MgBr₂. Among the separate ions present in the Dead Sea water are chloride (212.4 g/l), magnesium (40.65 g/l), sodium (39.15 g/l), calcium (16.86 g/l), potassium (7.26 g/l), bromide (5.12 g/l), sulfate (0.47 g/l), and bicarbonate (0.22 g/l). See, e.g., I. L. Schamberg, “Treatment of psoriasis at the Dead Sea,” Int J Dermatol, Vol. 17, No. 6 (1978), pp. 524-525; Paz and Shani, supra.

European Patent Application EP1074245A2 discloses use of mineral salt, in particular Dead Sea salts, as an active ingredient in a mouthwash to “assist in combating bacteria and gum irritation and inflammation”.

Essential oils have been used for the treatment of a variety of ailments since ancient times. The safety and efficacy of essential oils in dentistry have been reported in numerous clinical studies. See, e.g., Namrata Dagli et al., “Essential oils, their therapeutic properties, and implication in dentistry: A Review” J Int Soc Prev Community Dent. Vol. 5, No. 5 (2015), pp. 335-340.

The safety and potential for adverse effects from synthetic ingredients, not only for humans but also the larger ecosystem, have long been of concern. These issues were brought to the forefront by Rachel Carson, in her 1962 book, Silent Spring, which focused on the impact of pesticides, in particular DDT, on birds. A decade later, in 1973, the United States banned DDT. In that same year, manufacturers and producers of health foods and products began organic certification. Two years later, in 1975, Tom's of Maine introduced what it claimed to be the first mass-marketed “natural” toothpaste. The ensuing decades saw an explosive growth in demand for natural and organic products. By 1990, the organic industry had estimated sales of more than $1 billion. In 2006, Tom's of Maine was acquired by the Colgate-Palmolive Company. In 2015, Whole Foods had expanded to 365 stores and reported record revenues of almost $15.5 billion.

While natural personal care products have gained “mainstream” consumer acceptance, concerns remain. Many so-called “natural” products are not “natural”, and contain significant amounts of synthetic ingredients. Other products include “natural” ingredients at de minimis concentrations that do not provide health benefits; instead, natural ingredients are added to these products for purposes of “label copy”.

As access to the internet became more widespread, consumers took steps to publicly question what is natural, posting blogs and comments calling attention to what can be viewed as deceptive or misleading use of the word “natural.” Additionally, the internet has made do-it-yourself personal product recipes (for skincare, haircare, and oral care) available to consumers. See, e.g., http://www.healthyandnaturalworld.com/sage-and-sea-salt-homemade-toothpowder/ (¼ cup fresh sage leaves combined with ¾ cup sea salt); see also, http://www.sproutinghealthyhabits.com/homemade-natural-toothpaste/ (2 teaspoons of Dead Sea salt; 3 teaspoons of Himalayan pink salt; 2 teaspoons of ground sage; ⅓ cup of stevia powder; 7-8 tablespoons organic unrefined cold pressed coconut oil; 8 drops of tea tree essential oil; 40 drops spear[mint] essential oil; 15 drops of pepper[mint] essential oil; 5 teaspoons of sodium bentonite clay).

Access to a plethora of information on the internet is not, however, without risk. Website content is not subject to review and can be incomplete, inaccurate, or alarmist. Statements that a particular ingredient is “toxic” are often made without proper context. For example, a 1990 report issued by the US National Toxicology Program found “equivocal” evidence that fluoridated drinking water can cause osteosarcoma in male rats. However, exposure to fluoride has been associated with dental and skeletal fluorosis.

The present invention seeks to meet the long-felt but as yet unmet need for natural and naturally derived oral care products (in particular, dentifrices and mouthwashes) that contain safe and effective amounts of natural ingredients useful in cleaning and maintaining healthy, attractive teeth and gums. Products of the present invention are tailored to address fresher/cleaner breath.

Periodontal disease affects not only oral health. Recent research has identified potential linkage with systemic conditions such as cardiovascular disease, diabetes, adverse pregnancy outcomes, rheumatic arthritis, aspiration pneumonia and Chronic Obstructive Pulmonary Disease. Periodontal disease is also being investigated as a potential etiological factor in colorectal cancer, oral squamous cell carcinoma, pancreatic cancer and breast cancer.

### SUMMARY OF THE INVENTION

The present invention is directed to oral care products consisting essentially of (i) a sea salt, preferably Dead Sea salt; (ii) grapeseed oil; (iii) cayenne pepper oil; (iv) coconut oil; and (v) preferably, one or both of xylitol and/or aloe vera leaf juice; and uses of such compositions to reduce xerostomia.

### DETAILED DESCRIPTION OF THE INVENTION

A basic and novel characteristic of the oral care products of the present invention is the absence of: artificial colors, dyes or flavors; paraben or formaldehyde preservatives; bleaching agents (i.e., peroxides); sodium lauryl sulfate or sodium laureth sulfate; petroleum-derived glycerin; and genetically modified organisms (CMOs).

Another basic and novel characteristic of the oral care products of the present invention is non-cytotoxicity within the framework of ISO 10993-5 “Biological Evaluation of Medical Devices—Tests For In Vitro Cytotoxicity,” described in greater detail below.

In mouthwash embodiments, a further basic and novel characteristic of the oral care products of the present invention is the absence of ethyl alcohol and/or glycerin; preferably neither ethyl alcohol nor glycerin is present in the mouthwashes of the invention. Certain mouthwash embodiments of the present invention may sometimes be described as “alcohol free.” In labeling personal care and cosmetic products, the term “alcohol,” used by itself, is to be understood by the person having ordinary skill in the art as referring to ethyl alcohol. Accordingly, products labeled as “alcohol free” may contain other alcohols, including menthol or glycerol.

Collectively, ingredients listed above as absent from the oral care products of the present invention are referred to as “Excluded Ingredients.”

In certain preferred dentifrice embodiments, a further basic and novel characteristic of the oral care products of the present invention is the absence of fluoride and/or baking soda; preferably neither fluoride nor baking soda is present in the dentifrices of the invention. In those embodiments, fluoride and/or baking soda are to be considered as Excluded Ingredients. While dentifrice oral care products of the present invention are preferably not fluoridated, fluoride may be included in certain formulations within the scope of the invention to strengthen tooth enamel and remineralize teeth.

Accordingly, in describing and claiming oral compositions as “consisting essentially of” (i) a sea salt, preferably Dead Sea salt, (ii) grapeseed oil, (iii) cayenne pepper oil, and (iv) preferably, one or both of xylitol and/or aloe vera leaf juice it is meant that: (a) each of the following three ingredients are essential and, therefore, required component ingredients of the oral care products of the present invention: (i) sea salt, preferably Dead Sea salt, (ii) grapeseed oil, and (iii) cayenne pepper oil, are present in the oral care products; (b) preferably, one or both of xylitol and/or aloe vera leaf juice is/are present in the oral care products; and (c) Excluded Ingredients are not present in the oral care products.

“Xerostomia” is a condition in which a person has reduced saliva flow, and, therefore, reduced saliva volume in his/her mouth, causing the person to experience dryness in the mouth.

As used in the present application, an “essential oil” is a mixture of terpenic hydrocarbons, especially monoterpenes and sesquiterpenes, and oxygenated derivatives such as aldehydes, ketones, epoxides, alcohols, and esters.

“Non-cytotoxicity” of oral care products of the present invention is confirmed within the framework of ISO 10993-5:2009 based on the widely-used Trypan blue exclusion test. (Trypan blue is a colorant which stains dead cells, i.e., cells with loss of membrane integrity.) More particularly, Balb/c 3T3 clone A31 cells (ATCC CCL 163; 86th passage) are seeded in multi-well plates (24 wells, each 15.5 mm in diameter) at the starting density of 30,000 cells/cm2 in culture medium—Dulbecco's Modified Eagle Medium (DMEM)—supplemented with 10% (v/v) fetal calf serum (FCS). No antibiotics are used. Cultures are incubated at 37° C. in a humidified atmosphere containing 5% (v/v) CO₂, for 24 hours, and are examined with a microscope to verify a sub-confluent monolayer with less than de minimus alteration in morphology. Culture medium is withdrawn and replaced with a solution of one of the following: oral care products of the present invention at 5,000 μg/mL, as well as dilutions 1,500 μg/mL, 500 μg/mL and 150 μg/mL; a “negative” control (phenol, Chemical Abstract Service No. 108-95-2, 0.64 mg/mL); and a “positive” control (DMEM supplemented with 10% (v/v) FCS and 1% antibiotics (v/v). A positive control is defined as statistically significant (30% or greater) inhibition of cell growth as compared to the negative control.

Wells are incubated at 37° C. in a humidified atmosphere containing 5% (v/v) CO₂, for a 24-hour period. Photomicrographs are taken (320× magnification) showing the cell layer in contact with the negative control, the positive control and the oral care product of the present invention. Morphology and cell density of the cultures are observed. At the end of the incubation period, culture medium is removed. Cells are detached for two minutes using 250 μL trypsin (0.05% (w/v) in Hank's balanced solution Ca++ and Mg++ free supplemented with 1 mM EDTA. 250 μL of a Trypan blue solution at 0.2% (w/v) in 0.15 M NaCl and 10% (v/v) FCS are added. Cells are incubated for two minutes. Living cells (uncolored) are counted using a hemocytometer. Cell morphology and cell density of medium treated with 5,000 μg/mL of the oral care product of the present invention are observed to be comparable to those of negative control; neither shows statistically significant (30% or greater) inhibition of cell growth. In contrast, cells treated with the positive control show greater than 50% inhibition on cell growth.

Numbers used in describing quantities of ingredients and/or reaction conditions are to be understood as being modified in all instances by the term “about.”

Unless otherwise indicated, percentages, parts and ratios are to be understood as based upon the total weight of the composition.

Numerical ranges are meant to include numbers within the recited range, and combinations of subranges between, the given ranges. For example, a range from 1-5, includes 1, 2, 3, 4 and 5, as well as subranges such as 2-5, 3-5, 2-3, 2-4, 1-4, etc.

“At least one” means one or more, and also includes individual components as well as mixtures/combinations.

By the term “dentifrice” is meant a preparation for cleansing and polishing the teeth, that may, and preferably does provide one or more therapeutic benefits (as described below). As will be understood by the skilled artisan, a dentifrice (also referred to in the art and in this application as a “toothpaste”) may be formulated as a paste, gel or powder and is preferably applied with a toothbrush.

Dentifrice embodiments of the present invention are administered by brushing the teeth for at least 30 seconds, preferably for at least 60 seconds, most preferably for at least 120 seconds, at least once per day, in the evening prior to going to sleep, preferably with no eating or drinking within 30 minutes after administration.

By “brushing” is meant placing the bristles of a toothbrush in contact with the teeth, preferably at an angle of about 45 degrees to the gum line (where the gums and teeth meet), and moving the bristles in gentle, short strokes along the outer surfaces (cheek side), the inside surfaces (tongue side) and the chewing surfaces of all teeth. The strokes may be in a back-and-forth motion (side-to-side, or up-and-down) or a circular motion. After brushing, the dentifrice is expectorated.

Still more preferably, dentifrice embodiments of the present invention are administered by brushing the teeth for at least 30 seconds, preferably for at least 60 seconds, most preferably for at least 120 seconds, twice daily—once in the evening prior to going to sleep; and once in the morning after breakfast, preferably with no eating or drinking within 30 minutes after administration.

In each administration, about 0.25 grams of the dentifrice of the present invention, preferably at least about 0.5 grams is dispensed on to the bristles of a toothbrush and brushed onto the teeth. In certain preferred embodiments, the dose of toothpaste per administration ranges from about 0.4 to about 0.6 grams.

By the term “mouthwash” is meant a solution that is swished, preferably vigorously, around the mouth, and then expectorated, thereby cleaning the mouth and making the breath smell pleasant.

Mouthwash embodiments of the present invention are administered by swishing about 15-20 ml (about 0.5-0.75 fluid ounces) in the mouth, for a period of time sufficient to contact the teeth, the gums, the roof of the mouth and the tongue. Preferably, mouthwash is swished for at least 30 seconds, more preferably for at least 60 seconds, at least once per day, in the evening prior to going to sleep.

Still more preferably, mouthwash embodiments of the present invention are administered for at least 30 seconds, preferably for at least 60 seconds, twice daily—once in the evening, after brushing prior to going to sleep; and once in the morning after brushing after breakfast.

In especially preferred embodiments, a person practicing an oral care regimen in accordance with present invention (e.g., a consumer or a patient) brushes his/her teeth with a dentifrice of the invention for at least 30 seconds, more preferably for at least 60 seconds, and then swishes a mouthwash of the invention in his/her mouth for a period of at least 30 seconds, preferably at least 60 seconds—a period of time sufficient for the mouthwash to contact the teeth, the gums, the roof of the mouth and the tongue.

Persons practicing methods of the present invention are instructed to abstain from drinking or eating for at least 30 minutes after administering the oral care products of the invention; and, since oral care products of the invention are not intended for ingestion, to expectorate the oral care product after use (i.e., brushing in the case of a dentifrice, or swishing in the case of a mouthwash).

Sea salt is a mixture of inorganic salts from sea water or from inland bodies of salt water. Sea salt may be in the form of a precipitate (on the bottom of a marsh or salt pan or flat) or crystals that float on the surface of the water (known as fleur de sel).

One particularly preferred sea salt suitable for use in the oral care products of the present invention is Dead Sea salt, which is a mixture of natural hygroscopic minerals and micronutrients found in the Dead Sea and is comprised of sodium chloride, magnesium, potassium, and calcium chlorides and bromides. A non-limiting compositional analysis of Dead Sea salt versus common salt is presented in the table below:

| | Dead Sea | Common |
|---|---|---|
| | Salt (%) | Salt (%) |
| H₂O | 37.5 | 0.33 |
| MgCl₂ | 32.2 | 0.18 |
| KCl | 24.5 | 0.14 |
| NaCl | 5.6 | 99.2 |
| CaCl₂ | 6.23 | 0.15 |
| Br⁻ | 0.35 | 0.052 |
| Rb⁺ | 0.025 | — |
| Li⁺ | 1.0 | — |
| Fe³⁺ | 0.00203 | 0.00016 |
| Al³⁺ | 0.00037 | 0.000028 |
| SO₄²⁻ | 0.00916 | 0.0311 |
| Sr²⁺ | 0.00153 | 0.00047 |
| Mn²⁺ | 0.00023 | 0.0038 |

S. Halevy et al., J. Eur. Acad. Dermatol. Venereol., Vol. 9, pp. 237-242 (1997).

Another preferred sea salt suitable for use in the oral care products of the present invention is Himalayan salt, which is harvested from the Punjab Region of Pakistan, and is comprised of sodium chloride (about 95-98%), with about 2 to 3% polyhalite (potassium, calcium, magnesium, sulfur, oxygen, hydrogen), fluoride, iodine, and smaller amounts of other trace minerals.

Dead Sea salt is present in oral care products of the invention of the invention at a concentration of less than about 3%, preferably a concentration of from about 0.1% to 2%, still more preferably a concentration of from about 0.5% to about 1.5%.

Xylitol is the pentahydric alcohol that conforms to the formula:

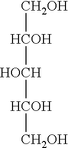

Xylitol may be, and preferably is, present in oral care products of the invention at a concentration of at least about 7.5%, more preferably at least about 10%, or at a dose of about 0.1 g/brushing or rinsing.

Aloe vera leaf juice useful in the present invention preferably contains (i) glycosides at a concentration of at least about 1%, preferably at least about 2%, and still more preferably at about 3%, as well as (ii) at least two, preferably three, anti-inflammatory agents selected from the group of anthraquinones, sterols, auxins and gibberellins and (iii) and immunomodulatory muccopolysachharides, preferably Acemannan.

Oral care products of the present invention contain one or more essential oils selected from the group consisting of spearmint oil, wintergreen oil, and peppermint oil.

Spearmint oil is the volatile oil obtained from the leaves of Mentha viridis (also known as Mentha spicata).

Wintergreen oil is the volatile oil obtained from the leaves of Gaultheria procumbens.

Peppermint oil is a volatile oil obtained from the whole plant Mentha piperita.

In one set of preferred embodiments, oral care products of the present invention include at least one essential oil in the genus Mentha, selected from Mentha piperita (peppermint) oil and Mentha viridis (spearmint) leaf oil.

In yet another preferred embodiment, oral care products of the present invention include Gaultheria procumbens (wintergreen) leaf oil.

In one even more preferred embodiment, oral care products of the present invention include peppermint oil and one of wintergreen oil or spearmint oil.

In another even more preferred embodiment, oral care products of the present invention include wintergreen oil and one of peppermint oil or spearmint oil.

In a still further even more preferred embodiment, oral care products of the present invention include spearmint oil and one of peppermint oil or wintergreen oil.

In especially preferred embodiments, the oral care products of the present invention contain spearmint oil, peppermint oil, and wintergreen oil.

Menthol, an alcohol that can be isolated from peppermint or other mint oils, can also be used in oral care products of the present invention.

Oral care products of the present invention also preferably include one or both of Ocimum basilicum (basil) oil and/or Eugenia caryophyllus (clove flower) oil.

In certain preferred embodiments of the invention, basil oil is present at a concentration of up to about 0.5%.

In certain preferred embodiments of the invention, clove oil is present at a concentration of at least about 0.005%. In other preferred embodiments of the invention, clove oil is present at a concentration of at least about 0.01%. In certain of these preferred embodiments, clove oil is preferably at a concentration of up to about 0.02%.

Other essential oils that may be included in oral care products of the present invention include Melaleuca alternifolia (tea tree) leaf oil, the oil distilled from the leaves of the Melaleuca alternifolia, and Zingiber officinale (ginger) root oil, which is obtained from the dried rhizomes of Zingiber officinale.

Dentifrice embodiments of the present invention may include mild abrasives (to remove debris and residual surface stains), humectants (to prevent water loss in the toothpaste), thickening products, also known in the art as binders (to stabilize the toothpaste formula), flavoring products (for taste) and detergents (to create foaming action).

Mild abrasives suitable for use in the toothpaste embodiments of the present invention include calcium carbonate, dehydrated silica gels, hydrated aluminum oxides, magnesium carbonate, phosphate salts and silicates. Silica, also called silicone dioxide, bentonite clay and hydrated silica are minerals. Some toothpastes of the present invention preferably contain hydrated silica.

Humectants that may be, and preferably are, ingredients in toothpastes of the present invention include glycerin, preferably vegetable glycerine, propylene glycol, and sorbitol.

Glycerin, a sugar alcohol that can be synthesized or obtained from natural sources, is an especially preferred humectant used in toothpastes of the invention.

Non-limiting examples of thickening products that may be, and preferably are included in toothpaste embodiments of the present invention include gums and colloids. Preferred colloids are of marine origin, even more preferably seaweeds.

Two preferred gums are xanthan gum and biosaccharide gum-1; both are polysaccharides derived from the fermentation of carbohydrates. Xanthan gum is derived from glucose or corn syrup. Biosaccharide gum-1 is derived from sorbitol.

Carrageenan, a polysaccharide hydrocolloid obtained from edible red seaweeds in the Gigartinaceae or Solieriaceae families, may be, and preferably is, present in toothpastes of the invention.

The present invention is directed to methods for relieving (i.e., reducing) the feeling of dryness in the mouth of a person having xerostomia by increasing saliva flow (and, thereby saliva volume). The invention may be practiced by administering an oral care product, a mouthwash or a toothpaste. The invention may also be practiced by administering a gel, that is comprised of (i) an “Active Ingredient Complex” consisting essentially of (a) a sea salt (preferably Dead Sea salt), (b) cayenne pepper oil, (c) grapeseed oil; and (d) coconut oil in (ii) a “Gel Carrier” comprised of at least one gelling agent known in the art that (xx) is generally recognized to be safe for oral use, (yy) does not readily dissolve in saliva, and (zz) does not react with, inactivate, or cause the degradation or loss of desired function of the Active Ingredient Complex.

Non-limiting examples of suitable gelling agents that may, and preferably are, components of the Gel Carrier, include: carboxypolymethylene, carboxymethyl cellulose, carboxypropyl cellulose, poloxamer, carrageenan, magnesium aluminum silicate (commercially-available under the tradename Veegum® from Vanderbilt Materials, LLC), carboxyvinyl polymers (including Carbomer), and natural gums such as gum karaya, xanthan gum, guar gum, gum arabic, gum tragacanth, and mixtures thereof.

Carbomers are a class of homopolymers of acrylic acid crosslinked with an allyl ether of pentaerythritol, an allyl ether of sucrose, or an allyl ether of propylene. One preferred Carbomer gelling agent is carboxypolymethylene, available from B. F. Goodrich Company under the tradename Carbopol®. Particularly preferred Carbopols include Carbopol® 934, 940, 941, 956 and mixtures thereof. Especially preferred is Carbopol® 956.

Another preferred gelling agent is polyvinylpyrrolidone. In certain embodiments polyvinylpyrrolidone is of a molecular weight in a range of 1,000,000 to 1,500,000. In certain embodiments, polyvinylpyrrolidone has molecular weight of about 50,000 to about 300,000.

Additional gelling agents that are suitable for use in the whitening strips of the present invention include celluloses (hydroxy ethyl or propyl cellulose, ethyl cellulose) and Polyox® water-soluble resins available from Dow Corning (Midland, Mich.).

Polyox® resins are nonionic, high molecular weight water-soluble poly (ethylene oxide) polymers with molecular weights ranging from 100,000 to about 8,000,000.

Gantrez® copolymers—monoalkyl esters of poly(methyl vinyl ether/maleic acid) with varying ester groups—available from Ashland are also suitable to thicken the whitening compositions and promote adhesion between the strip and teeth surfaces.

### EXAMPLES

The following examples illustrate compositions and methods of practicing of the present invention in some of its embodiments; the examples should not be construed as limiting the scope of the invention. Other embodiments will be apparent to one skilled in the art from consideration of the specification and examples. It is intended that the specification, including the examples, is considered exemplary only without limiting the scope and spirit of the invention.

Some of the examples illustrate preferred embodiments of the invention. Variations of these preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, unless otherwise indicated herein or otherwise clearly contradicted by context, the inventions include all modifications and equivalents of the subject matter disclosed and recited in the claims appended hereto as permitted by applicable law.

### Dentifrice Examples

Dentifrices of the present invention are formulated for use by persons having xerostomia and may contain the following ingredients at the following concentrations:

Dead Sea salt is present in toothpastes of the invention at a concentration of less than about 3%, preferably from about 0.5% to 2%, most preferably from about 0.75% to about 1.5%.

Coconut oil is present in toothpastes of the present invention at a concentration of from about 0.001% to about 5%, preferably from about 0.005% to about 1%, and most preferably from about 0.09% to about 0.9%.

Grape seed oil is present in toothpastes of the invention at a concentration of from about 0.01% to about 1%, preferably from about 0.03% to about 0.5%, and most preferably from about 0.07% to about 0.4%.

Cayenne pepper oil is present in toothpastes of the present invention at a concentration of from about 0.01% to about 1.0%, preferably from about 0.02% to about 0.3%, and most preferably from about 0.03% to about 0.2%.

Xylitol may be, and preferably is, present in toothpastes of the present invention at a concentration of at least 10% or at a dose of 0.1 g/brushing.

Aloe vera leaf juice may be, and preferably is, present in toothpastes of the present invention at a concentration of at least about 10%, preferably at least about 20%, and most preferably at least about 40%.

Mentha piperita (peppermint) oil is present in toothpastes of the invention at a concentration of less than 1%, preferably less than about 0.075%, more preferably at a concentration of less than about 0.05%, and even more preferably at a concentration of about 0.03%.

Mentha viridis (spearmint) oil is present in toothpastes of the invention, preferably at a concentration of up to about 4%, preferably up to about 2%, more preferably up to about 1%, and still more preferably less than about 0.5%.

Gaultheria procumbens (wintergreen) leaf oil is present in toothpastes of the invention, preferably at a concentration of up to about 1%, preferably from about 0.25% to about 0.5%.

In some preferred toothpaste embodiments, the weight ratio of wintergreen oil to peppermint oil is about 10:1.

In other preferred toothpaste embodiments, the weight ratio of spearmint oil to peppermint oil is about 3:1.

In still other preferred toothpaste embodiments, the weight ratio of wintergreen oil to spearmint oil is about 3:1.

In one preferred toothpaste embodiment, the weight ratio of wintergreen to peppermint is from about 6:1 to about 5:1.

In another preferred toothpaste embodiment, the weight ratio of spearmint to peppermint is about 5:3.

In still another preferred toothpaste embodiment, the weight ratio of wintergreen to spearmint is about 4:1.

Basil oil (also known as Ocimum tenuiflorum oil) is present in toothpastes of the invention, preferably at a concentration of up to about 0.05%.

Clove oil is present in toothpastes of the invention, preferably at a concentration of up to about 0.05%.

Glycerin is present in toothpastes of the present invention at a concentration of from about 2.5% to about 20%, preferably from about 5.0% to about 15%.

Carrageenan may be, and preferably is, present in toothpastes of the invention, preferably at a concentration of at least about 0.05%. preferably about 0.1%. Even more preferably, carrageenan is food-grade.

Xanthan gum may be, and preferably is present in toothpastes of the invention, preferably at a concentration of at least about 0.10.

Titanium dioxide may be present in certain toothpastes of the invention; when present, titanium dioxide is present at a concentration of up to about 0.6%.

Hydrated silica may be, and preferably is, present in toothpastes of the invention, at a concentration of from about 10% to about 25%.

Toothpastes of the present invention contain a foaming anionic other than sodium lauryl sulfate, preferably sodium methyl cocoyl taurate or sodium lauroyl sarcosinate. In certain preferred embodiments, sodium methyl cocoyl taurate is present in toothpastes of the invention at a concentration of up to about 2%.

### Mouthwash Examples

Dead Sea salt is present in mouthwashes of the present invention at a concentration of from about 0.5% to about 5%, preferably from about 0.75% to about 3%, and most preferably from about 1% to about 2%.

Coconut oil is present in mouthwashes of the present invention at a concentration of from about 0.001% to about 5%, preferably from about 0.1% to about 0.7%, and most preferably from about 0.15% to about 0.6%.

Grape seed oil is present in mouthwashes of the present invention at a concentration of from about 0.01% to about 1%, preferably from about 0.03% to about 0.5%, and most preferably from about 0.07% to about 0.3%.

Cayenne pepper extract is present in mouthwashes of the present invention at a concentration of from about 0.01% to about 1.0%, preferably from about 0.02% to about 0.3%, and most preferably from about 0.03% to about 0.15%.

Xylitol may be, and preferably is, present in mouthwashes of the present invention at a concentration of from about 5% to about 30%, preferably from about 7% to about 15%, and most preferably from about 8% to about 12%.

Aloe vera leaf juice may be, and preferably is, present in mouthwashes of present invention at a concentration of from about 10% to about 90%, preferably from about 20% to about 85%, and most preferably from about 50% to about 70%.

Clove flower oil is preferably present in mouthwashes of the present invention at a concentration of from about 0.005% to about 0.075%, preferably from about 0.01% to about 0.04%, and most preferably from about 0.01% to about 0.03%.

Basil oil is preferably present in mouthwashes of the present invention at a concentration of from about 0.005% to about 0.5%, preferably from about 0.01% to about 0.2%, and most preferably from about 0.02% to about 0.1%.

Peppermint oil is present in mouthwashes of the present invention at a concentration of from about 0.005% to about 0.12%, preferably from about 0.01% to about 0.1%, and most preferably from about 0.02% to about 0.09%.

Spearmint oil is present in mouthwashes of the present invention at a concentration of from about 0.01% to about 1%, preferably from about 0.02% to about 0.17%, and most preferably from about 0.05% to about 0.15%.

Wintergreen oil is present in mouthwashes of the present invention at a concentration of from about 0.03% to about 1%, preferably from about 0.05% to about 0.5%, and most preferably from about 0.1% to about 0.45%.

### Study of Oral Biofilm and Dry Mouth

A 3-leg study is conducted on ten patients each of whom are clinically diagnosed with xerostomia. In two legs, subjects use a test or control product for xerostomia intervention; in the third leg, they use no intervention for xerostomia. The “test product” is a mouthwash of the invention as described above and formulated for use in the treatment of dry mouth. The control product is Biotene™ Dry Mouth Oral Rinse.

The study aims are as follows:
(i) Use 2-Tone™ disclosing solution (Young Dental Manufacturing, Earth City, Mo.) combined with digital photography and image analysis to quantify biofilm presence at baseline and after 7-day use of a test and a control product, each vs no xerostomia treatment product with a 1-week washout between the 3 legs of the study.
(ii) Use conventional clinical indices Plaque Index (PI), Gingival Index (GI) and Sulcus Bleeding Index (mSBI) to quantify gingival health at baseline and after 7-day use of a test and a control product vs no xerostomia treatment product with a 1-week washout between the 3 legs of the study.
(iii) Quantify salivary volume, pH and pH-buffering performance at baseline and after each 7-day study leg.
(iv) Document subject self-evaluation of dentinal sensitivity at baseline and after 7-day use of a test product and a control agent vs. no xerostomia treatment product with a 1-week washout between each of the 3 legs of the study.
(v) Document subject self-evaluation for dry mouth using a standardized questionnaire at baseline and after 7-day use of a test product and a control product vs no xerostomia treatment product with a 1-week washout between the 3 legs of the study (e.g., after the first study leg and again after the second study leg).

Subjects are randomized with regard to sequence of the 3 legs of the study. Salivary volume, pH and buffering capacity are determined by asking the subject to pool saliva in the floor of their mouth for a 5-minute duration, then expectorate the saliva into a sterile collecting cup. During the leg incorporating test and control product use, subjects rinse with the test/control product. Saliva pooling/expectorant is collected twice, after ten minutes and after 40 minutes. During the “no test product” leg, subjects rinse with water instead. Additionally, subjects complete a standardized self-evaluation questionnaire for dry mouth at each visit.

The following results are observed:

Saliva volume doubles at the end of the test and control study legs, representing a statistically significant increase in each case (p<0.05). In the study leg with no intervention, saliva production does not change significantly (p>0.1).

PH values do not differ significantly between study legs and time-points (p>0.05).

1. An oral care product consisting essentially of
a. a sea salt;
b. cayenne pepper oil;
c. grapeseed oil; and
d. coconut oil.

2. The oral care product of claim 1 wherein the sea salt is Dead Sea salt.

3. The oral care product of claim 2 wherein the sea salt is Dead Sea salt, which is present at a concentration of from about 0.5% to about 3.0%.

4. An oral care product according to claim 3 that contains one or both of xylitol or aloe vera leaf juice.

5. An oral care product according to claim 4 wherein the oral care product contains at least one of peppermint oil, wintergreen oil, or spearmint oil.

6. An oral care product according to claim 3 that contains an essential oil of one or both of basil or clove flower.

7. An oral care product according to claim 6 wherein xylitol is present at a concentration of at least 5%.

8. An oral care product according to claim 7 wherein the oral care product is a dentifrice and contains vegetable glycerin and at least one surfactant, wherein the at least one surfactant (a) is coconut derived and (b) is not sodium lauryl sulfate or sodium laureth sulfate.

9. An oral care product according to claim 8 wherein the at least one surfactant is sodium methyl cocoyl taurate.

10. An oral care product according to claim 9 further comprising at least one non-abrasive powder that is a hydrated silica.

11. A method for increasing saliva volume in an oral cavity of a person having xerostomia comprising the step of administering at least once daily the oral care product of claim 7 for at least 30 seconds.

12. The method of claim 11 wherein the step of administering the oral care product is performed at least twice daily, each time for at least 30 seconds.

13. The method of claim 12 wherein the step of administering the oral care product is performed (i) a first time in the evening, prior to going to sleep, and (ii) a second time in the morning, after breakfast.

14. The method of claim 12 wherein after a person administers the oral care product the person abstains from eating or drinking for a period of at least about 30 minutes.

15. The method of claim 11 wherein saliva volume is increased by at least about 50%.

16. The method of claim 15 wherein saliva volume is increased by at least about 100%.
